(19) United States
(12) Patent Application Publication
(10) Pub. No.: US 2023/0248719 A1
(43) Pub. Date: Aug. 10, 2023

(54) STERILIZED FORMULATIONS

(71) Applicant: HARM REDUCTION THERAPEUTICS, INC., Pittsburgh, PA (US)

(72) Inventor: Richard Mannion, Furlong, PA (US)

(21) Appl. No.: 18/008,611

(22) PCT Filed: Jun. 8, 2021
(86) PCT No.: PCT/US21/36341

§ 371 Date: Dec. 6, 2022

Related U.S. Application Data
(60) Provisional application No. 63/036,207, filed on Jun. 8, 2020.

(51) Int. Cl. A61K 31/485; A61K 9/00

(52) U.S. Cl. CPC A61K 31/485; A61K 9/0043

(57) ABSTRACT

Disclosed in certain embodiments is a method of sterilization comprising subjecting a pharmaceutical composition comprising naloxone or a pharmaceutically acceptable salt thereof to a temperature and time sufficient for sterilization, the method having an FO value of from 8 to 15.

### FIELD OF THE INVENTION

The present application is directed to stabilized formulations of naloxone or pharmaceutically acceptable salts thereof.

### BACKGROUND

Certain pharmaceutical compositions that are not properly sterilized can lead to negative clinical outcomes to a patient such as microbial infection or a reduction in potency of active agent due to degradation. Accordingly, it is important that pharmaceutical compositions are properly sterilized and maintained sterile from manufacture, through transport and storage, to the ultimate administration to a patient in need. This is especially true with opioid antagonist compositions that are intended to inhibit or reverse opioid overdose as any reduction in available active agent or microbial contamination can result in increased morbidity and mortality to patients in need.

There continues to exist a need in the art for sterilized pharmaceutical formulations of naloxone or pharmaceutically acceptable salts thereof and methods of preparation and treatment thereof.

### OBJECTS AND SUMMARY

It is an object of certain embodiments of the present invention to provide sterilized naloxone formulations and methods or preparation and methods of treatment.

In certain embodiments, the present invention is directed to a pharmaceutical composition comprising an aqueous formulation comprising naloxone or a pharmaceutically acceptable salt thereof in an amount equivalent to 30 mg naloxone HCl per ml application fluid, the composition having an unknown degradation content of no more than 0.5% per individual unknown degradant, no more than 0.33% per individual unknown degradant, or no more than 0.25% per individual unknown degradant.

In certain embodiments, the present invention is directed to a method of sterilization comprising subjecting a pharmaceutical composition comprising naloxone or a pharmaceutically acceptable salt thereof to a temperature and time sufficient for sterilization, the method having an F0 value of from 8 to 15.

In certain embodiments, the present invention is directed to a pharmaceutical composition comprising naloxone or a pharmaceutically acceptable salt thereof, wherein the composition is subjected to a temperature and time sufficient for sterilization, the method having an F0 value of from 8 to 15 prepared according to any of the preceding claims.

In certain embodiments, the present invention is directed to a method of treating opioid overdosing, the method comprising intranasally administering to a patient in need thereof a pharmaceutical composition comprising naloxone or a pharmaceutically acceptable salt thereof, wherein the composition is subjected to a temperature and time sufficient for sterilization, the method having an F0 value of from 8 to 15 prepared according to any of the preceding claims.

### DETAILED DISCLOSURE

Certain sterilization techniques require the application of heat such as autoclaving, which is a sterilization process that utilizes steam. Unfortunately, the autoclaving process may result in degradation of the active agent if the thermal exposure exceeds a certain threshold. By virtue of certain embodiments of the present invention, a heat sterilization process is disclosed that does not provide an excessive amount of degradation.

In certain embodiments, the present invention is directed to a method of sterilization comprising subjecting a pharmaceutical composition comprising naloxone or a pharmaceutically acceptable salt thereof to a temperature and time sufficient for sterilization, the method having an F0 value of from 8 to 15. In alternative embodiments, the present invention provides an F0 value of from 8 to 14; an F0 value of from 9 to 13; an F0 value of from 10 to 14; an F0 value of from 12 to 14; an F0 value of from 11 to 13; an F0 value of from 12 to 15; or an F0 value of about 12.

In certain embodiments of the present invention, the maximum temperature of the sterilization process is from about 110° C. to about 130° C.; from about 115° C. to about 125° C.; or about 121° C.

In certain embodiments of the present invention, the time at the maximum temperature of the sterilization process is from about 3 minutes to about 15 minutes; from about 4 minutes to about 8 minutes; or about 5 minutes.

In certain embodiments, the sterilization process utilizes moist heat or steam (e.g., in an autoclaving process).

In certain embodiments, the naloxone is in the form of the hydrochloride salt.

In certain embodiments, the pharmaceutical composition is an aqueous solution and can have a pH, e.g., of ≤5.5.

In certain embodiments, the composition does not include a permeability enhancer or a microbiological preservative.

In certain embodiments, the concentration of the naloxone or pharmaceutically acceptable salt thereof is equivalent to between 20 mg naloxone HCl per ml fluid and 60 mg HCl per ml fluid; between 30 mg naloxone HCl per ml fluid and 50 mg naloxone HCl per ml fluid; or equivalent to about 30 mg naloxone HCl per ml.

In certain embodiments, the present invention is directed to pharmaceutical compositions that may be prepared as disclosed herein.

In certain embodiments, the present invention is directed to a method of treating a patient in need of naloxone therapy (e.g., opioid overdose, opioid withdrawal syndrome, alcohol dependence or constipation), the method comprising administering (e.g., intranasally or parenterally) to a patient in need thereof a pharmaceutical composition as disclosed herein.

In certain embodiments, the process and compositions disclosed herein provide a pharmaceutical composition comprising naloxone or a pharmaceutically acceptable salt thereof that has an unknown degradation content (e.g., RRT˜0.503 or RRT˜0.586) of no more than 0.5% per individual unknown degradant, no more than 0.33% per individual unknown degradant, no more than 0.25% per individual unknown degradant, no more than 0.10% per individual unknown degradant or no more than 0.08% per individual unknown degradant.

In certain embodiments, the process and compositions disclosed herein provide a pharmaceutical composition comprising naloxone or a pharmaceutically acceptable salt thereof that has a noroxymorphone degradation content of no more than 1.0%, no more than 0.5%, no more than 0.1%, or no more than 0.07%.

In certain embodiments, the process and compositions disclosed herein provide a pharmaceutical composition comprising naloxone or a pharmaceutically acceptable salt thereof that has a 10-α-hydroxynaloxone or pseudonaloxone degradation content of no more than 0.5%, no more than 0.1%, no more than 0.05%, or below the limit of detection.

In certain embodiments, the process and compositions disclosed herein provide a pharmaceutical composition comprising naloxone or a pharmaceutically acceptable salt thereof that has a noroxymorphone degradation content of no more than 1.0%, no more than 0.5%, no more than 0.1%, or no more than 0.05%

In certain embodiments, the process and compositions disclosed herein provide a pharmaceutical composition comprising naloxone or a pharmaceutically acceptable salt thereof that has a total unknown degradation content of no more than 1.0%, no more than 0.5%, no more than 0.3% or no more than 0.2%.

In certain embodiments, the present invention is directed to a pharmaceutical composition comprising an aqueous formulation comprising naloxone or a pharmaceutically acceptable salt thereof in an amount equivalent to 30 mg naloxone HCl per ml application fluid, the composition having an unknown degradation content of no more than 0.5% per individual unknown degradant, no more than 0.33% per individual unknown degradant, or no more than 0.25% per individual unknown degradant.

In certain embodiments, the pharmaceutical compositions disclosed herein are in an amount of 100 μl.

In certain methods of treatment disclosed herein, the method comprises intranasally administering to one nostril of a patient in need thereof, and optionally repeating administration until an effective amount of naloxone is reached in the systemic circulation of the patient to provide a therapeutic effect.

The term “F0” is defined as the number of equivalent minutes of steam sterilization at 121° C. delivered to a load. For example, if a cycle has an F0 value of 12, the sterilization effectiveness of that cycle is equal to 12 minutes at 121° C. regardless of the process temperature and time used in the cycle.

In certain embodiments, the intranasal dosage form is a nasal spray, a nasal mucoadhesive dosage form or a Mucosal Atomizer Device, all of which can easily be administered not only by trained medical personnel but also by a medically untrained subject. In certain embodiments, the device is capable of functioning in a supine position as well as in upright position.

A nasal spray may be a syringe-driven spraying device or a pump-driven spraying device. In certain embodiments, the device is pre-primed. The formulation which is used in the intranasal dosage form may be a solution, a suspension or a nasal gel/gel-like formulation.

In certain embodiments, pharmaceutical excipients used in the intranasal formulations of the present invention include absorption/permeability enhancers, binders and carriers. Other excipients that can be used include tonicity agents, a buffers, solvents, co-solvents, viscosity agents and gelling agents.

In certain embodiments, the formulation is a liquid and may be, e.g., solutions, suspensions or emulsions. In one embodiment, the formulation may comprise water or water-propylene glycol solutions. Additional ingredients in liquid preparations may include, for example, antimicrobial preservatives, such as benzalkonium chloride, methylparaben, sodium benzoate, benzoic acid, phenyl ethyl alcohol, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and mixtures thereof; and a suspending agent such as microcrystalline cellulose, carboxymethylcellulose sodium NF, polyacrylic acid, magnesium aluminum silicate, xanthan gum, and mixtures thereof.

In certain embodiments, the formulation comprises one or more excipients selected from water and NaCl.

In certain embodiments, the formulation is substantially free of antimicrobial preservatives.

In certain embodiments, the formulation comprises one or more excipients selected from water, NaCl, benzalkonium chloride, sodium edetate, disodium edetate, and hydrochloric acid.

In certain embodiments, the formulation comprises water, NaCl, benzalkonium chloride, disodium edetate, and hydrochloric acid.

In certain embodiments, the formulation comprises an isotonicity agent; a preservative; a stabilizing agent; an amount of an acid sufficient to achieve a pH or 3.5-5.5; and an amount of water sufficient to achieve a final volume of about 100 μL.

In certain embodiments, the formulation comprises between about 0.2 mg and about 1.2 mg of an isotonicity agent; between about 0.005 mg and about 0.015 mg of a preservative; between about 0.01 mg and about 0.05 mg of a stabilizing agent; an amount of an acid sufficient to achieve a pH or 3.5-5.5; and an amount of water sufficient to achieve a final volume of about 100 ml.

In certain embodiments, the isotonicity agent is NaCl; the preservative is benzalkonium chloride; the stabilizing agent is disodium edetate; and the acid is hydrochloric acid.

In certain embodiments, the formulation comprises about 0.74 mg NaCl; about 0.01 mg benzalkonium chloride; about 0.2 mg disodium edetate; an amount of hydrochloric acid sufficient to achieve a pH or 3.5-5.5; and an amount of water sufficient to achieve a final volume of about 100 μL.

In some embodiments, the formulation is storage-stable for at least 6 months, at least 12 months, at least 24 months, about 6 months, about 12 months or about 24 months at about 25° C. and about 60% relative humidity.

The formulations disclosed herein may be used in the treatment of an opioid overdose symptom, e.g., selected from respiratory depression, postoperative opioid respiratory depression, altered level consciousness, miotic pupils, cardiovascular depression, hypoxemia, acute lung injury, aspiration pneumonia, sedation, and hypotension. The opioid overdose can be caused by, e.g., codeine, morphine, methadone, fentanyl, oxycodone, hydrocodone, hydromorphone, oxymorphone, meperidine, propoxyphene, tapentadol, tramadol, opium, heroin, or salts thereof.

### EXAMPLES

Specific embodiments of the invention will now be demonstrated by reference to the following examples. It should be understood that these examples are disclosed solely by way of illustrating the invention and should not be taken in any way to limit the scope of the present invention.

A naloxone nasal formulation was prepared as set forth below in Table 1.

TABLE 1
Composition of Naloxone Nasal Spray, Solution

| | Quality | Quantity per | Quantity per | |
|---|---|---|---|---|
| Component | Standard | dose (mg) | vial (mg) | Function |
| Naloxone | USP/NF | 3.30¹ | 4.125 | Active ingredient |
| hydrochloride | | | | |
| dihydrate | | | | |
| Trisodium citrate | USP/NF | 0.74 | 0.92 | Buffer |
| dihydrate | | | | |
| Sodium chloride | USP/NF | 0.19 (or | 0.24 | Tonicity |
| | | lower) | | |
| Hydrochloric acid | USP/NF | q.s. | q.s. to pH 4 | pH adjustment |
| Sodium hydroxide | USP/NF | q.s. | q.s. to pH 4 | pH adjustment |
| Nitrogen | USP/NF | q.s. | q.s. | Deaeration |
| Purified water | USP/NF | q.s. to 100.9² | q.s. to 126.1³ | Solvent |

¹Equivalent to 3.00 mg naloxone hydrochloride anhydrous and 2.70 mg naloxone base
²Based on nominal delivered volume of 100 μl
³Based on nominal overfill volume of 125 μl to ensure nominal delivered volume of 100 μl

An autoclave cycle which subjected the product to less thermal stress with the target of reducing degradation products (especially unknown degradants) at the end of the cycle was utilized. Three development cycles were ran and the product tested for degradation product. The product was hand filled into suitable containers and with no nitrogen headspace.

### Cycle 1-15-Minutes at 121.0° C. Air Over Pressure Moist Heat Process

Cycle 1 Air-Over-Pressure (AOP) Steam Sterilization Run Conditions

| | | | | | | JACKET |
|---|---|---|---|---|---|---|
| | | | | RATE | HOLD TIME | TEMP. |
| LOOP | PHASE | SET POINT | | (psia/min) | (min:sec) | (° C.) |
| 0 | Heat Up* | 115.0° | C. | 2.0° C. | 05:00 | 118.0 |
| 0 | Heat Up* | 121.0° | C. | 1.5° C. | 05:00 | 118.0 |
| 0 | Sterilization* | 121.0° | C. | 0.0 | 15:00 | 118.0 |
| 0 | Air Pulsed | 45.0 to 45.5 | psia | N/A | 15:00** | <118.0 |
| | Drying 1 | | | | | |
| 0 | Air Pulsed | 45.0 to 45.5 | psia | N/A | 15:00*** | <118.0 |
| | Drying 2 | | | | | |
| 0 | Exhaust | 14.7 | psia | 2.0 | 00:00 | <118.0 |

| | | | | | |
|---|---|---|---|---|---|
| 0 | Equalization | ambient | 1.0 | N/A | <118.0 |

*Programed for AOP cycle with Support Pressure, Jacket Temp. set at 118.0° C. and Steam/Air Mixture Enabled, with heat-up and sterilization controlled with Load probe. With 15.0 PSIA Support Pressure during heat-up and sterilization.
**Final Pressurization Temperature of 90.0° C.
***Final Pressurization Temperature of 63.0° C.

### Cycle 1 Thermal Mapping Data

| | TC01 | TC02 | TC03 | TC04 |
|---|---|---|---|---|
| Location | Center Right | Center Left | Camber | Drain |
| Min | 121.1° C. | 121.0° C. | 121.0° C. | 112.1° C. |
| Max | 121.2° C. | 121.2° C. | 121.2° C. | 120.7° C. |
| Avg | 121.2° C. | 121.1° C. | 121.1° C. | 113.2° C. |
| F⁰ | 32.0 | 31.0 | | |

Cycle 2 — 5-minutes at 121.0° C. Air Over Pressure Moist Heat Process

Cycle 2 AOP Steam Sterilization Run Conditions

| | | | | | HOLD | JACKET |
|---|---|---|---|---|---|---|
| | | | | RATE | TIME | TEMP. |
| LOOP | PHASE | SET POINT | | (psia/min) | (min:sec) | (° C.) |
| 0 | Heat Up* | 115.0° | C. | 2.0° C. | 05:00 | 118.0 |
| 0 | Heat Up* | 121.0° | C. | 1.5° C. | 05:00 | 118.0 |
| 0 | Sterilization* | 121.0° | C. | 0.0 | 5:00 | 118.0 |
| 0 | Air Pulsed Drying 1 | 45.0 to 45.5 | psia | N/A | 15:00** | <118.0 |
| 0 | Air Pulsed Drying 2 | 45.0 to 45.5 | psia | N/A | 15:00*** | <118.0 |
| 0 | Exhaust | 14.7 | psia | 2.0 | 00:00 | <118.0 |

| | | | | | |
|---|---|---|---|---|---|
| 0 | Equalization | ambient | 1.0 | N/A | <118.0 |

*Programed for AOP cycle with Support Pressure, Jacket Temp. set at 118.0° C. and Steam/Air Mixture Enabled, with heat-up and sterilization controlled with Load probe. With 15.0 PSIA Support Pressure during heat-up and sterilization.
**Final Pressurization Temperature of 90.0° C.
***Final Pressurization Temperature of 63.0° C.

Cycle 2 Thermal Mapping Data

| | TC01 | TC02 | TC03 | TC04 |
|---|---|---|---|---|
| Location | Center Right | Center Left | Camber | Drain |
| Min | 121.3° C. | 121.0° C. | 120.7° C. | 104.8° C. |
| Max | 121.4° C. | 121.1° C. | 120.8° C. | 105.4° C. |
| Avg | 121.3° C. | 121.1° C. | 120.8° C. | 105.1° C. |
| F₀ | 28.4 | 26.8 | | |

### Cycle 3-3-Minutes at 121.0° C. Air Over Pressure Moist Heat Process

Cycle 3 AOP Steam Sterilization Run Conditions

| | | | | | | JACKET |
|---|---|---|---|---|---|---|
| | | | | RATE | HOLD TIME | TEMP. |
| LOOP | PHASE | SET POINT | | (psia/min) | (min:sec) | (° C.) |
| 0 | Heat Up* | 121.0° | C. | 2.0° C. | 01:00 | 118.0 |
| 0 | Sterilization* | 121.0° | C. | 0.0 | 3:00 | 118.0 |
| 0 | Air Pulsed Drying 1 | 45.0 to 45.5 | psia | N/A | 10:00** | <118.0 |
| 0 | Exhaust | 14.7 | psia | 2.0 | 00:00 | <118.0 |

| | | | | | |
|---|---|---|---|---|---|
| 0 | Equalization | ambient | 1.0 | N/A | <118.0 |

*Programed for AOP cycle with Support Pressure, Jacket Temp. set at 118.0° C. and Steam/Air Mixture Enabled, with heat-up and sterilization controlled with Load probe. With 15.0 PSIA Support Pressure during heat-up and sterilization.
**Final Pressurization Temperature of 90.0° C. (phase controlled by either time or temperature - whichever gets to set-point last) Heat-up and Air Pulsed Drying changed to only one phase each.

### Cycle 3 Thermal Mapping Data

| | TC01 | TC02 | TC03 | TC04 |
|---|---|---|---|---|
| Location | Center Right | Center Left | Camber | Drain |
| Min | 121.2° C. | 121.1° C. | 121.1° C. | 119.4° C. |
| Max | 121.4° C. | 121.3° C. | 121.2° C. | 119.4° C. |
| Avg | 121.3° C. | 121.2° C. | 121.1° C. | 119.7° C. |
| Data Logger | 14.5 | 14.2 | | |
| Total Cycle F₀ | | | | |

| | |
|---|---|
| Sterilizer RTD F₀ at end | 13.3 |
| of Cycle | |
| Sterilizer RTD F₀ at end | 4.9 |
| of Heat-Up | |
| Sterilizer RTD F₀ at end | 7.74 |
| of Sterilization | |

All of Cycles 1-3 gave no degradation products greater than LOD (0.02%). Cycles 2 and 3 had no upward stopper movement (stoppers started at a target of 16.0) (see table below for stopper and degradation product data)

### LOD was 0.02% and LOQ 0.06%

| | | Cycle 1 | Cycle 2 | Cycle 3 |
|---|---|---|---|---|
| Measurement | 1 | 16.5 | 16.2 | 15.9 |
| (Distance between vial and | 2 | 16.8 | 15.9 | 15.8 |
| stopper) | 3 | 16.7 | 15.7 | 15.8 |
| Limit: 15.7-16.3 mm | 4 | 16.4 | 15.5 | 15.6 |
| | 5 | 16.5 | 15.5 | 15.5 |
| | 6 | 16.3 | 15.6 | 15.8 |
| | 7 | 16.4 | 15.6 | 15.8 |
| | 8 | 16.1 | 15.6 | 15.8 |
| | 9 | 16.1 | 15.6 | 15.9 |
| | 10 | N/A | 15.5 | 15.8 |

Degradants (%)

| | | | |
|---|---|---|---|
| Noroxymorphone (N1) | ND | ND | ND |
| 10-α-Hydroxynaloxone (N2) | ND | ND | ND |
| 7,8-Didehydronaloxone (N3) | ND | ND | ND |
| 2,2′-Bisnaloxone (N4) | ND | ND | ND |
| Naloxone N-oxide (N5) | ND | ND | ND |
| 3-O-Allylnaloxone (N6) | ND | ND | ND |
| RRT ~0.314-RRT ~0.315 | ND | ND | ND |
| RRT ~0.497 | ND | ND | ND |
| RRT ~0.581 | ND | ND | ND |
| RRT ~1.152 | ND | ND | ND |
| RRT ~2.067 | ND | ND | ND |
| Total | ND | ND | ND |

Reference: CR to TTP-HBE-M0028, Phase MD BK 01 Page 3; BK 02, LDR 06, 07
ND = Not Detected

Three 10 L batches were manufactured with the composition detailed in Table 1. Solution was filled into borosilicate glass micro vials with chlorobutyl stoppers and a low oxygen/nitrogen headspace. Each batch was autoclaved separately using a cycle based on Cycle 3 but with 2 minutes added to the “sterilization” phase of the cycle to give a total of 5 minutes, and tested for degradation products.

AOP Sterilization Full-cycle Conditions

| | SET POINT | | | | | JACKET |
|---|---|---|---|---|---|---|
| PHASE | ° C. | PSIA | RATE | | HOLD TIME | TEMP. |
| Heat Up* | 121.0 | N/A | 2.0° | C./minute | 1.0-minute | 118.0° C. |
| Sterilization* | 121.0 | N/A | N/A | | 5.0-minute | 118.0° C. |
| AOP | 90.0 | 45.0 to 45.5 | N/A | | 10.0-minute** | <118.0° C. |
| Exhaust | 0.0 | 14.7 | 2.0 | PSIA/minute | 0.0-minute | <118.0° C. |
| Equalization | N/A | ambient | 1.0 | PSIA/minute | 0.0-minute | <118.0° C. |

*Programmed for AOP cycle with Support Pressure, Jacket Temp. set at 118.0° C. and Steam/Air Mixture Enabled, with heat-up and sterilization controlled with Load probe. With 15.0 PSIA Support Pressure during heat-up and sterilization.
**Final Pressurization Temperature of 90.0° C. is satisfied.

The F0 for the three batches were 14.0, 13.4, 14.3.
Degradation Product results were:

| Degradation Product | 1^{st} Batch | 2^{nd} Batch | 3^{rd} Batch |
|---|---|---|---|
| Noroxymorphone | 0.07 | 0.07 | 0.06 |
| 10-α-Hydroxynaloxone | ND | ND | ND |
| 2,2′-Bisnaloxone | ND | ND | ND |
| (Pseudonaloxone) | | | |
| Naloxone N-oxide | NQ | NQ | 0.05 |
| Unknown # 1 | 0.08 | 0.07 | 0.06 |
| (RRT ~0.503) | | | |
| Unknown # 2 | 0.06 | 0.05 | 0.05 |
| (RRT ~0.586) | | | |
| Total Degradation Products | 0.20 | 0.18 | 0.20 |

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A method of sterilization comprising subjecting a pharmaceutical composition comprising naloxone or a pharmaceutically acceptable salt thereof to a temperature and time sufficient for sterilization, the method having an FO value of from 8 to 15.

2. The method of claim 1, having an F0 value of from 9 to 13.

3. The method of claim 1, having an F0 value of from 10 to 14.

4. The method of claim 1, having an F0 value of from 12 to 14.

5. The method of claim 1, having an F0 value of from 12 to 15.

6. The method of claim 1, having an F0 value of about 12.

7. The method of claim 1, wherein the maximum temperature is from about 110° C. to about 130° C.

8. The method of claim 1, wherein the maximum temperature is from about 115° C. to about 125° C.

9. The method of claim 1, wherein the maximum temperature is about 121° C.

10-22.(canceled)

23. A pharmaceutical composition prepared according to claim 1.

24. A method of treating opioid overdosing, the method comprising intranasally administering a patient in need thereof a pharmaceutical composition according to claim 23.

25. A pharmaceutical composition comprising an aqueous formulation comprising naloxone or a pharmaceutically acceptable salt thereof in an amount equivalent to 30 mg naloxone HCl per ml application fluid, the composition having an unknown degradation content of no more than 0.5% per individual unknown degradant, no more than 0.33% per individual unknown degradant, or no more than 0.25% per individual unknown degradant.

26. The pharmaceutical composition of claim 25, suitable for nasal or parenteral administration.

27. The pharmaceutical composition of claim 25, suitable for nasal administration.

28. The pharmaceutical composition of any of claims 25-27, wherein the composition has a pH of ≤5.5.

29. The pharmaceutical composition of claim 25, wherein the composition does not include a permeability enhancer.

30. The pharmaceutical composition of claim 25, wherein the composition does not include a microbiological preservative.

31. The pharmaceutical composition of claim 25, wherein the composition is in an amount of 100 μl.

32. A method of treating opioid overdosing, the method comprising intranasally administering to a patient in need thereof a pharmaceutical composition according to claim 25.

33. The method of claim 32, comprising administering the composition in one nostril of the patient and optionally repeating administration until an effective amount of naloxone is reached in the systemic circulation of the patient to counter the effect of the opioid.
